(19) United States
(12) Patent Application Publication
(10) Pub. No.: US 2024/0280505 A1
(43) Pub. Date: Aug. 22, 2024

(54) Apparatus and method for inspecting filled containers and their filling product

(71) Applicant: KRONES AG, Neutraubling (DE)

(72) Inventors: Ines BRADSHAW, Regensburg (DE); Patrick ENGELHARD, Elsendorf (DE); Valentin BECHER, Donaustauf (DE)

(21) Appl. No.: 18/571,701

(22) PCT Filed: May 24, 2022
(86) PCT No.: PCT/EP2022/064131

§ 371 Date: Dec. 18, 2023

(30) Foreign Application Priority Data
Jun. 17, 2021 (DE) 10 2021 115 729.3

(51) Int. Cl. G01N 21/90; B65B 57/00; B65B 61/28; G01N 21/84

(52) U.S. Cl. CPC G01N 21/90; B65B 57/00; B65B 61/28; G01N 2021/8411; G01N 2021/845

(57) ABSTRACT

An installation for producing containers filled with a liquid, and in particular beverage containers, has at least one transport device which transports containers along a predetermined transport path, a filling device which fills the containers, a closure device which closes the filled containers, and an inspection device that inspects the filled and closed containers, wherein the inspection device is arranged downstream of the closure device along the transport path. The inspection device is configured to output at least one measured value that is characteristic of a composition of the filling product filled into the containers.

The present invention relates to an apparatus and a method for inspecting containers filled with a liquid, and in particular for inspecting these liquids. Numerous apparatuses and methods for inspecting beverage containers are known from the prior art in the beverage manufacturing industry. A wide variety of parameters are determined, such as foreign body detection, filling level detection and the like. In addition, it would also be desirable in the prior art to not only detect defects in the containers, such as foreign bodies in a liquid, or a relevant filling level. It would also be advantageous to receive information about the contents of the container or the composition of the content.

An example of a filling installation for filling beverages is known from US 2006/0037286 A1. U.S. Pat. No. 10,501,302 B2 discloses a method for recognizing an error condition within the scope of beverage filling.

A container filling arrangement is known from EP 3 433 204 B1, wherein a measuring sensor system is provided that is connected to the filling vessel or a circuit line, wherein a beverage component supply is controlled as a function of an output signal of the measuring sensor. In this installation, the medium to be filled into the containers is checked. However, it is not possible to check the filled containers in this way.

The person skilled in the art is therefore faced with the object of providing an apparatus and a method allowing the content of filled containers to be checked. These objects are achieved by the subject matters of the independent claims. Advantageous embodiments and developments are the subject matter of the dependent claims.

An installation according to the invention for producing liquid containers, and in particular beverage containers, has a transport device that transports containers along a predetermined transport path and a filling device which fills the containers and a closure device which closes the filled containers (with a closure), and an inspection device which inspects the filled and closed containers, wherein said inspection device being arranged downstream of the closure device along the transport path.

According to the invention, the inspection device is suitable and intended for outputting at least one measured value that is characteristic of a composition of the filling product filled into the containers.

It is therefore proposed that this inspection device inspects and in particular analyzes the liquid filled into the containers. In a preferred embodiment, the transport device transports the containers past the inspection device and/or the inspection device is integrated into the installation. The inspection device preferably allows an inspection of the beverage containers to be produced, in a working operation of the installation. The inspection device is preferably suitable and intended for online inspection of the containers.

The containers described here can in particular be bottles and beverage bottles, in particular plastic bottles or glas bottles.

The installation can furthermore have a production device which produces the containers, such as a blow molding machine, in particular a stretch blow molding machine.

Preferably, the containers are at least partially and preferably completely transparent to radiation and in particular to light in the visible and infrared wavelength range.

The transport device is particularly preferably suitable and intended for transporting the containers at a transport speed that is between 0.05 m/s and 3 m/s.

In a further preferred embodiment, the inspection device is arranged in a straight transport path portion of the containers. However, it would also be conceivable for the transport apparatus to transport the containers along a circular or circular-segment-shaped transport path and for the inspection to take place in this region.

In a further preferred embodiment, the installation has several inspection devices. These can, for example, be arranged one behind the other along the transport path of the containers.

In a further preferred embodiment, a settling section, which causes the liquid within the container to come to rest and/or to be substantially at rest, is arranged between the closure device and the inspection device.

Within the scope of the invention, it is therefore proposed to carry out a contactless and/or optical and/or, as described in more detail below, in particular spectroscopic measurement and in particular online measurement of the product itself through an (in particular transparent) bottle or packaging.

The advantage is that, in this way, filling processes in which mixing takes place in a filling valve or only in the container can be monitored, i.e., after the actual filling process.

The filling apparatus is preferably suitable and intended for filling a beverage that is composed of a plurality of components (e.g., water and syrups) into the containers.

According to the current prior art, no 100% quality control takes place for such technologies. The product quality can only be examined offline in a laboratory (for example, by gas chromatography). This can be changed by the application of the present invention and in particular by the application of an online spectroscopic measurement. A spectroscopic measurement is therefore preferably carried out.

A continuous 100% online and process monitoring for each individual container is thus made possible, since the quality control takes place directly on the container. The advantage of the application of spectroscopic methods for monitoring foodstuffs is supplemented by the measurement from the outside through the container.

In a further preferred embodiment, the installation has a further inspection device which is suitable and intended for inspecting the containers. It is particularly preferred for this further inspection device to be arranged upstream of the inspection device described here in the transport direction of the containers. However, it would also be possible to arrange it downstream of the inspection device described here, which inspects the filling product.

This further inspection device can check the container itself, for example defects in the container, the presence of foreign bodies in the container, or a filling level of the liquid, and the like.

Particularly preferably, the inspection device is configured and intended for carrying out the inspection from the outside through the container.

In a preferred embodiment, the filling device has a plurality of filling elements that fill the containers. These filling elements can be arranged, for example, on a rotatable carrier, by means of which the containers are also transported. The transport device described above can in turn be composed of many sub-devices, such as transport starwheels. These filling elements preferably each have individually controllable filling valves.

Furthermore, the filling device can have a first reservoir for storing a first component of a beverage and a second reservoir for storing a second component of a beverage, wherein said reservoirs each being able to be brought into flow connection with the filling elements.

In a preferred embodiment, the inspection device is suitable and intended for inspecting the containers during their transport and in particular during their movement. In this way, online measurement of the container content is possible.

In a preferred embodiment, the above-mentioned measured value is characteristic of a property of the liquid which is selected from a group of properties that include the presence of predetermined constituents or substances in the liquid, a concentration of a substance in the liquid, a concentration of components of the liquid, the presence of gaseous phases in the liquid, and the like.

It is possible for the inspection device to output qualitative values, such as values that are characteristic of the presence of a specific substance, or quantitative values, such as information about a concentration. Which of these values should be output in each case can also depend on the user. It may, for example, be important to the user to determine whether the constituents of the liquid to be filled are present in the correct concentration ratio. Furthermore, the user may be interested in determining whether specific foreign bodies and/or foreign substances (which may, for example, come from earlier uses of the same container) are present in the liquid.

The inspection device is preferably suitable and intended for determining the detection of trace substances in the liquids in a range of up to μg per liter and below. The inspection device is preferably able to prevent, for example, aroma carryover or to check the beverage that has already been filled into the closed bottle. Preferably, a final product control of beverages in filled and closed bottles (i.e., not the liquid directly) and in particular a positive/negative control of the filled beverage should be made possible. Furthermore, the quality of the beverage with regard to dilution or carryover of other liquids is preferably checked by the invention.

Within the scope of extensive tests, it was possible to determine that the sensors used are suitable for inspection. The bottles that are filled with the beverage usually serve as measuring cells themselves. Initial evaluations and assessments of the spectral results were performed via visual assessments.

For lower detection limits and more precise evaluation as well as for the later calibration of specific contaminants, the installation preferably has an evaluation device and in particular a software-based evaluation device. With appropriate algorithms, significantly lower detection limits can be achieved than with a purely visual assessment. The evaluation device preferably evaluates the measured results using artificial intelligence.

Preferably, the determination of at least one measured value and particularly preferably the determination of a plurality (or all) of the measured values of a (in particular each) liquid to be detected and/or analyzed, and/or in particular the determination of data from the measured values generated by the inspection device or data derived therefrom is based on (computer-implemented) machine learning methods, preferably machine learning methods based on at least one (artificial) neural network. Such a neural network can be designed, for example, as a deep neural network (DNN) and/or a convolutional neural network (CNN) and/or a recurrent neural network (RNN).

Classes for classifying the analysis results (for example, of specific wavelength-dependent maxima or minima of a spectrum) are preferably predetermined. The classes are preferably stored in an external storage device (in particular described below) and can be updated by retrieving these stored classes. The classes can preferably be compared with at least one reference spectrum.

Preferably, in order to determine the at least one linked parameter and preferably to determine the plurality of linked parameters (in particular by evaluating a spectral analysis), (at least) a data set relating to a liquid and comprising the sensor data (or data derived therefrom) determined (by means of the sensor device) and/or the determined defect size(s) and/or the parameters (selected and/or set at the time of treatment) of the filling device filling the particular container in question or of a filling element of this filling device is processed by means of an artificial neural network.

Preferably, in order to determine the at least one linked parameter and preferably to determine the plurality of linked parameters (in particular by the spectral analysis device), (at least) a plurality of data sets (described in the paragraph above) relating to a plurality of detected liquids are processed by means of the artificial neural network. The data sets relating to a specific liquid, in particular a liquid that deviates from a target state, can each relate to data sets relating to liquids of a specific filling device. The plurality of data sets relating to a specific liquid preferably relates to at least two different filling devices and preferably to a plurality of different (reference) filling devices and/or to specific containers filled with reference liquids. The plurality of data sets is preferably stored in the (external) storage device, in particular the (external) server.

The data sets, in particular the measured values (or data derived therefrom) and/or the defect size(s) and/or the parameters (selected and/or set at the time of treatment) of the filling device filling the observed container are preferably supplied as input variables to the artificial neural network. The artificial neural network preferably forms the input variables onto output variables depending on a parameterizable processing chain, wherein at least one linked parameter and preferably a plurality of linked parameters of a (predetermined) filling device are selected as the output variable(s).

The artificial neural network is preferably designed as a deep neural network (DNN), in which the parameterizable processing chain has a plurality of processing layers, and/or as a convolutional neural network (CNN) and/or a recurrent neural network (RNN).

The artificial neural network is preferably trained using predetermined training data, wherein the training parameterizing the parameterizable processing chain. Preferably, the training data used are data sets relating to the data sets described above relating to at least one liquid, which was filled in particular by a specific filling device and preferably a plurality of different filling devices, or of filled containers. A neural network trained in this way is preferably used. Training is preferably carried out by means of supervised learning. However, it would also be possible to train the artificial neural network by means of unsupervised learning, reinforcement learning, or stochastic learning.

In a further preferred embodiment, the measured value to be output is selected from a group of measured values. This means that the user can select what should be examined and, for example, whether quantitative or qualitative tests should be carried out.

In a further preferred embodiment, the inspection device has a radiation device which directs radiation onto the containers, and a radiation detector device which detects radiation passing through the containers.

Preferably, an irradiation direction in which the radiation device radiates onto the container is at an angle different from 0° to a longitudinal direction of the container. This means that the radiation device preferably radiates through a container wall into the container and the radiation exits again on the opposite side of the container. The radiation device particularly preferably directs the radiation onto a main body of the container or onto a shoulder region of the container.

This means that measurements are particularly preferably carried out using a transmitted light method and, as mentioned above, the containers themselves serve as measuring cells.

Preferably, a beam path of the radiation passing through the container is selected in such a way that little or no obstruction occurs due to a label of the container. Preferably, there is no additional influence by further changes to the bottle geometry.

In a further preferred embodiment, the installation has a spectrometer and/or a monochromator and/or the installation has a radiation device having a variable emission spectrum, in particular an emission spectrum that is variable with regard to the wavelengths. In this way, a beam analysis can be carried out.

A beam analysis is particularly preferably carried out in a wavelength range between 300 nm and 3000 nm.

In a further, preferred embodiment, the radiation device is a laser device, in particular a laser device that can be tuned with regard to the emission, such as a dye laser or an OPO (optical parametric oscillator).

In a further preferred embodiment, the installation has an evaluation device that evaluates the measured values output by the inspection device. It is possible for this evaluation device to compare the measured values with reference values in order to, for example, search for or determine specific spectral or spectroscopic properties. As mentioned above, artificial intelligence can be used.

In a further preferred embodiment, the installation has a discharge device which is arranged downstream of the inspection device in the transport direction of the containers and which is suitable and intended for excluding or discharging containers from the transport path, taking into account a result or a measured value (or a plurality of measured values) output by the inspection device. For example, containers that have been identified as defective due to their content can be excluded from further processing.

In a further preferred embodiment, the installation has at least one further inspection device which is suitable and intended for inspecting the filled containers. For example, it is possible to check for defects in the container, for example for a plastic distribution of the plastic container, for foreign bodies within the container, or for the filling level, and the like.

In a further preferred embodiment, the installation has an assigning device which is suitable and intended for assigning a further treatment element, such as a filling member, to an inspected container, which filling member has filled this container. In this way, a targeted intervention in the installation is possible, for example in order to switch off individual filling members in response to such inspection results or to modify them with regard to the filling of the beverage.

The present invention is furthermore directed to a method for producing liquid containers and in particular beverage containers, wherein a transport device transports containers along a predetermined transport path and a filling device fills the containers and a closure device closes the filled containers, and an inspection device inspects the filled and closed containers. This inspection device is arranged downstream of the closure device along the transport path.

According to the invention, the inspection device outputs at least one measured value that is characteristic of a composition of the filling product filled into the containers.

The filling device is preferably suitable and intended for filling different or several components of a beverage into each container.

In a further preferred method, the inspection device inspects the containers during movement and/or during transport of the containers. In a further preferred method, a radiation device directs radiation onto the containers and a detector device detects radiation passing through the containers.

The inspection device particularly preferably carries out a spectral analysis of the radiation passing through the containers. In a further preferred method, the measured values recorded by the inspection device are compared with comparative values.

Further advantages and embodiments can be seen in the accompanying drawings:

In the drawings:

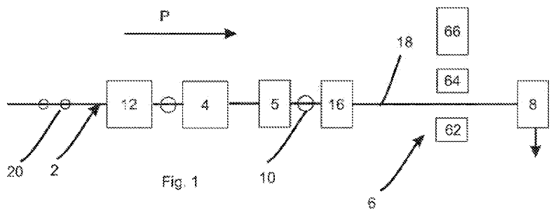

FIG. 1 shows a rough schematic representation of an installation according to the invention.

FIG. 1 is a schematic representation of an installation according to the invention for producing beverage containers. Here, plastic preforms 20 are supplied to a forming device 12, such as a blow molding machine, and formed into containers 10. These containers are filled by means of a filling device 4 and closed by means of a closure device 5. An optional inspection device 16 can be connected to this closure device, which inspection device for example checks specific criteria, for example whether the filling level corresponds to predetermined criteria or whether there are foreign bodies in the container.

A transport section 18, which serves to bring the medium in the container to rest, is preferably connected to this inspection device. In its entirety, the transport device which transports the containers along a transport path P is denoted by 2.

Reference sign 6 denotes the inspection device which serves to analyze the medium in the container, i.e., in particular the beverage. This inspection device has a radiation device 62 and a radiation detector device 64, wherein the containers being transported between these devices 62, 64. Reference sign 66 denotes an evaluation device that analyzes the radiations recorded by the detector device 64.

Reference sign 8 denotes a discharge device which is suitable and intended for discharging containers, and in particular defective containers, from the transport path in response to signals from the inspection devices 16 and/or 6.

The applicant reserves the right to claim all features disclosed in the application documents as essential to the invention, provided that they are novel over the prior art individually or in combination. It is also pointed out that features which can be advantageous in themselves are also described in the individual figures. The person skilled in the art will immediately recognize that a particular feature described in a figure can be advantageous even without the adoption of further features from this figure. Furthermore, the person skilled in the art will recognize that advantages can also result from a combination of several features shown in individual or in different figures.

1. An installation for producing containers filled with a liquid, comprising at least one transport device configured to transport containers along a predetermined transport path, comprising a filling device configured to fill the containers, comprising a closure device configured to close the filled containers, and an inspection device that inspects the filled and closed containers, wherein said inspection device is arranged downstream of the closure device along the transport path,
wherein
the inspection device is configured to output at least one measured value which is characteristic of a composition of the filling product filled into the containers, wherein the installation has a spectrometer and/or a monochromator and/or a radiation device having a variable emission spectrum.

2. The installation according to claim 1,
wherein
the inspection device is configured to inspect for inspecting the beverage containers during their transport.

3. The installation according to claim 1,
wherein
the measured value is characteristic of a property of the liquid which is selected from a group of properties consisting of the presence of predetermined constituents or substances, a concentration of a substance, and a concentration of components of the liquid.

4. The installation according to claim 1.
wherein
the measured value to be output is selected from a group of measured values.

5. The installation according to claim 1.
wherein
the inspection device has a radiation device configured to direct radiation on-to the containers and a radiation detector device configured to detect the radiation passing through the containers.

6. (canceled)

7. The installation according to claim 1,
wherein
the installation has an evaluation device configured to evaluate the measured values output by the inspection device.

8. The installation according to claim 1.
wherein
the installation has a discharge device which is arranged downstream of the inspection device in the transport direction of the containers and which is configured to discharge containers from the transport path, taking into account a result output by the inspection device.

9. The installation according to claim 1.
wherein
the installation has at least one further inspection device which is configured to inspect the filled containers.

10. The installation according to claim 1,
wherein
the installation has an assigning device which is configured to inspect a filling member to an inspected container, which filling member has filled this container, wherein the installation has a spectrometer and/or a monochromator and/or a radiation device having a variable emission spectrum.

11. A method for producing liquid containers, wherein a transport device transports containers along a predetermined transport path and a filling de-vice fills the containers and a closure device closes the filled containers, and an inspection device inspects the filled and closed containers, wherein said inspection device is arranged downstream of the closure device along the transport path.
wherein
the inspection device outputs at least one measured value which is characteristic of a composition of the filling product filled into the containers.

12. The method according to claim 11.
wherein
the inspection device inspects the containers during movement of the containers.

13. The method according to claim 11.
wherein
a radiation device directs radiation onto the containers and a detector device detects radiation passing through the containers.

14. The method according to claim 11,
wherein
the inspection device carries out a spectroscopic measurement.

15. The method according to claim 11,
wherein
the measured values recorded by the inspection device are compared with comparative values.
